(19) United States
(12) Patent Application Publication
(10) Pub. No.: US 2003/0144708 A1
(43) Pub. Date: Jul. 31, 2003

(54) Methods and apparatus for retarding stomach emptying for treatment of eating disorders

(76) Inventor: Warren L. Starkebaum, Plymouth, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/150,430

(22) Filed: May 17, 2002

Related U.S. Application Data
(60) Provisional application No. 60/352,681, filed on Jan. 29, 2002.

(51) Int. Cl. A61N 1/18

(52) U.S. Cl. 607/40

(57) ABSTRACT

Methods and systems for treating patients suffering from eating disorders, e.g. obesity, through the delivery of electrical stimulation directly or indirectly to the pylorus of a patient in an effective stimulation regimen to substantially close the pylorus lumen to inhibit emptying of the stomach. The stimulation electrodes are applied directly to or immediately adjacent to the muscles layers of the pyloric sphincters or are situated in operative relation to the splanchnic nerve that innervates the pyloric sphincter. Stimulation can be delivered continuously 24 hours per day or can be halted at meal times to enable passage of chyme through the pylorus lumen at such times. Alternatively, stimulation is delivered following events related to peristalsis, ingestion or stomach emptying to induce a feeling of satiety.

### RELATED APPLICATION

This application claims priority to provisional U.S. Application Ser. No. 60/352,681, filed Jan. 29, 2002.

### FIELD OF THE INVENTION

The present invention pertains to methods and systems for treating patients suffering from eating disorders particularly obesity by selectively electrically directly or indirectly stimulating the muscle layers of the pyloric sphincter to close or restrict the pylorus lumen, e.g., at programmed eating times of day or upon activation by the patient or upon detection of eating related to detection of GI tract signals indicating stomach emptying.

### BACKGROUND OF THE INVENTION

Obesity among adults and children is an increasing problem due generally to increases in caloric intake coupled with declines in exercise levels. Morbid obesity among the same population is also increasing as these habitual tendencies are coupled with physiologic conditions of certain individuals predisposed to obesity that may not fully understood in a given case. The primary treatment has always involved behavioral change involving dietary restraints to reduce caloric intake coupled with aerobic and anaerobic exercise routines or physical therapy regimens to increase caloric expenditure, resulting in a net caloric reduction. Diet and exercise plans fail since most individuals do not have the discipline to adhere to such rigorous discipline. Consequently, the marketplace is flooded with resurrected or new dietary supplements and ethical (or prescription) and patent (or nonprescription) drugs or other ingestible preparations promoted as capable of suppressing appetite or inducing satiety (i.e., the satisfied feeling of being full after eating) or of “burning” fat.

In general, these techniques for treating compulsive overeating/obesity have tended to produce only a temporary effect. The individual usually becomes discouraged and/or depressed in the course of the less radical therapies primarily focused on behavioral change after the initial rate of weight loss plateaus and further weight loss becomes harder to achieve. The individual then typically reverts to the previous behavior of compulsive overeating and/or indolence.

In advanced or extreme cases, treatment of obesity has included wiring the jaws shut for a time. Liposuction (suction lipectomy) procedures are also sometimes employed to remove adipose tissue from obese patients. Liposuction also enjoys wide application for cosmetic reshaping of the anatomy, particularly the abdomen, hips, thighs and buttocks of non-obese persons. Patients undergoing liposuction and jaw wiring may enjoy their lower weight and bulk for a time, but eventually typically regain the excised or lost weight and volume.

More radical surgical approaches are also commonly performed alone or sometimes in combination to restrict food intake or to limit absorption of nutrients in morbidly obese patients. Surgical approaches to restrict food intake include gastric banding, gastric bypass, and vertical-banded gastroplasty to decrease the size of the stomach to reduce the amount of food the stomach can hold and/or to delay the emptying of the stomach. Surgical approaches to limit nutrient absorption typically connect the stomach to the lower part of the small intestine thereby bypassing the duodenum and part of the small intestine.

Although these surgical approaches work well for some patients, many patients experience serious unpleasant side effects that, together with the risk, recuperation pain, and expense of such major surgery, discourage their widespread adoption. Risks attendant to restricting food intake include failure or weakening of the staple or suture lines causing leakage of stomach contents into the abdomen or pouch stretching. Bypass procedures carry the risk of creating nutritional imbalances because, for example, Fe and Ca are absorbed mostly in the duodenum. Bypass procedures can cause “dumping syndrome” in which stomach contents move too rapidly through the remaining small intestine causing nausea, vomiting, or diarrhea. Patients may be required to use special foods or supplements and medications to manage these complications. The need to treat morbidly obese patients is so great that about 50,000 such procedures costing in excess of one billion dollars are done each year in the United States despite these risks and complications,

The gastro-intestinal tract, also called the alimentary canal, is a long tube through which food is taken into the body and digested. The alimentary canal begins at the mouth, and includes the pharynx, esophagus, stomach, small and large intestines, and rectum. In human beings, this passage is about 30 feet (9 meters) long.

Small, ring-like muscles, called sphincters, surround portions of the alimentary canal. In a healthy person, these muscles contract or tighten in a coordinated fashion during eating and the ensuing digestive process, to temporarily close off one region of the alimentary canal from another region.

For example, a muscular ring called the lower esophageal sphincter surrounds the opening between the esophagus and the stomach. The lower esophageal sphincter (or LES) is a ring of increased thickness in the circular, smooth muscle layer of the esophagus. Normally, the lower esophageal sphincter maintains a high-pressure zone between 15-30 mm Hg above intragastric pressures inside the stomach.

When a person swallows food, muscles of the pharynx push the food into the esophagus. The muscles in the esophagus walls respond with a wavelike contraction called peristalsis. The lower esophageal sphincter relaxes before the esophagus contracts, and allows food to pass through to the stomach. After food passes into the stomach, the lower esophageal sphincter constricts to prevent the contents from regurgitating into the esophagus.

The pylorus shown in FIG. 1 is a specialized region at the junction of the antrum and the duodenal bulb that serves the physiologic role of a sieve to regulate the passage of chyme from the stomach. The pylorus possesses unique neural and smooth muscle characteristics as well as a distinct shape that distinguishes it from the antrum and the duodenum. A pyloric sphincter surrounds the pylorus lumen into the duodenum and is formed of proximal and distal smooth muscle loops joined by a muscular torus on the lesser curvature. The characteristics and function of the pylorus are described in the Textbook of Gastroenterology, Volume 1, T. Yamada ed., Lippincott, 1995, pp. 188-191, in “Sensory Nerves of the Intestines: Role in Control of pyloric Region of Dogs” by G. Tougas et al. (Sensory Nerves and Neuropeptides in Gastroenterology, M. Costa et al. ed. Plenum Press New York, 1991, pp.199-211), and in “Neuromuscular Differentiation of the Human Pylorus” by K. Schulze-Delrieu et al. (GASTROENTEROLOGY 1983:84, pp. 287-92). K. Schulze-Delrieu et al refer to the proximal smooth muscle loop and the distal smooth muscle loop as the “intermediate sphincter” and “distal sphincter” respectively.

Food is ingested until a feeling of satiety is induced and/or the stomach is distended. During ingestion and for a time thereafter, the smooth muscle layers of the pyloric sphincter are contracted to restrict the pylorus lumen and keep food in the stomach until it is liquefied. The ingested food bolus is propelled aborally mixed and ground in the antrum against the closed pylorus, and then retro-propelled orally into the more proximal corpus. The muscles of the stomach rhythmically churn ingested food and digestive juices into a mass called chyme. The, stomach muscles contract peristaltic waves triggered by a gastric pacemaker region shown in FIG. 1 and move downward or reterograde toward the pylorus and mix and sheer the food into chyme while the pylorus lumen is closed. After the ingested food is ground into chyme, the pyloric sphincter relaxes in concert with antral motor activity of each peristaltic wave and lets some chyme pass into the duodenum. The pylorus lumen is small enough to function as a sieve to only let minute food particles enter the duodenum in the absence of active contraction of the pyloric sphincter.

FIG. 1 also illustrates electrogastrogram (EGG) signals that cause the depicted peristaltic wave contraction of the stomach wall. Such EGG signals normally originate in the putative pacemaker region near the junction along the greater curvature of the proximal one third or fundus and the distal two thirds of the stomach comprising the corpus and antrum. The EGG signals include slow waves that normally appear every 10-30 seconds or at a frequency of 2-6 cycles per minute (cpm), typically about 3 cpm, and propagate along the stomach wall in a characteristic pattern down to the corpus and pyloric antrum. The slow waves cause the stomach wall to rhythmically contract and move food remaining in the stomach toward the pylorus and duodenum in the peristaltic wave depicted in FIG. 1. The peristaltic wave contraction functions to create shear on the stomach contents and thus break the contents down into smaller particles that can pass through the pylorus lumen.

For example, 3 cpm slow waves are illustrated in FIG. 1 that can be sensed at three locations B, C, D but are not sensed at location A as long as the stomach is functioning normally. The three sensed EGG signals at locations B, C, D exhibit normal timed synchronization. During a peristaltic contraction, the slow waves further feature a higher voltage, high frequency action or spike potential. Each slow wave shown in FIG. 1 at B, C and D features a corresponding high frequency action potential shortly thereafter. The slow waves, as discussed above, typically have a frequency of 3 cpm. The higher frequency action potentials, however, typically have a frequency of between 100-300 Hz.

The peristaltic wave contractions are not conducted through the pylorus to the duodenum. The duodenum rhythmically contracts in a similar fashion under the control of a separate duodenal pacemaker and a rate of about 12 cpm. The relaxation of the pyloric sphincter is independent of the duodenal contractions and is independent of but timed to peristaltic contractions of the antrum.

Pyloric obstructions occur in some infants and occasionally in adults wherein ingested food cannot pass through the pylorus lumen in sufficient quantity to provide adequate nutrition. The stomach fills and its contents are then regurgitated. Infants suffer malnutrition and failure to thrive unless surgical procedures are undertaken to correct the obstruction.

In some individuals, either the regular rhythmic peristaltic contractions do not occur or the regular rhythmic electrical depolarizations do not occur or both do not occur. In each of these situations the movement of food may be seriously inhibited or even disabled. One such condition that occurs as a result of generalized peritonitis or shock is often called “paralytic ilius” that sometimes occurs after abdominal surgery. Another condition that is often called “gastroparesis” is a chronic gastric motility disorder in which there is delayed gastric emptying of solids or liquids or both from the stomach. Symptoms of gastroparesis may range from early satiety and nausea in mild cases to chronic vomiting, dehydration, and nutritional compromise in severe cases. Similar motility disorders occur in the other organs of the GI tract, although by different names.

Diagnosis of gastroparesis is based on-demonstration of delayed gastric emptying of a radio-labeled solid meal in the absence of mechanical obstruction. Gastroparesis may occur for a number of reasons. Management of gastroparesis involves four areas: (1) prokinetic drugs, (2) anti-emetic drugs, (3) nutritional support, and (4) surgical therapy (in a very small subset of patients.) Gastroparesis is often a chronic, relapsing condition; 80% of patients require maintenance anti-emetic and prokinetic therapy and 20% require long-term nutritional supplementation. Other maladies such as tachygastria or bradygastria can also hinder coordinated muscular motor activity of the GI tract, possibly resulting in either stasis or nausea or vomiting or a combination thereof.

The undesired effect of these conditions is a reduced ability or complete failure to efficiently propel intestinal contents down the digestive tract. This results in malassimilation of liquid or food by the absorbing mucosa of the intestinal tract. If this condition is not corrected, malnutrition or even starvation may occur. Moreover nausea or vomiting or both may also occur. Whereas some of these disease states can be corrected by medication or by simple surgery, in most cases treatment with drugs is not adequately effective, and surgery often has intolerable physiologic effects on the body.

The concept of electrically stimulating the gastro-intestinal tract to restore its proper function and alleviate paralytic ilius originated many years ago, and one early approach is disclosed in commonly assigned U.S. Pat. No. 3,411,507. The ′507 patent discloses a system for gastro-intestinal stimulation which uses an electrode positioned on a nasogastric catheter and an electrode secured to the skin over the abdomen. In operation, the nasogastric catheter is inserted into the patient's stomach while the patient is lying down such that the electrode is positioned in close proximity to the pylorus either in the antrum or in the duodenum. Electrical stimulation is delivered for the first five seconds of every minute until peristaltic activity in the antrum is initiated. The stimulation process is discontinued after the first bowel movement. It is asserted in the ′507 patent that the induced “peristaltic waves cross the pylorus and are carried down to the duodenum” and activate its pacemaker area. However, this assertion, and the efficacy of the stimulation, has been contested by later researchers (Sarna et al., infra). The ′507 patent system was a short-term device that was only useful for patients in a hospital setting, and particularly non-ambulatory patients to facilitate emptying of the stomach and duodenum. The disclosed system and method of the ′507 patent did not enjoy widespread acceptance.

It is possible to sense both the slow waves and the higher frequency action potentials and process the sensed waves to indicate the state of the stomach at that moment. This is especially useful to thereby determine or detect the presence or absence of peristaltic contraction within the stomach. EGG sense amplifiers of the type described in commonly assigned U.S. Pat. No. 6,083,249, for example coupled to sense electrodes at one or more of the locations B, C, D in the manner described therein can differentiate between the slow waves and the spike potentials. Thus, it is possible to sense spike activity characteristic of peristalsis and to generate a spike sense event on detection of each spike potential. The amplitude and frequency detection thresholds of such sense amplifiers are programmable and can be adjusted to the particular characteristics of the spike potentials in a given patient in a manner well known in the art and the cardiac pacing art.

The sensed EGG signals have been employed typically to detect slow waves recurring at a lower rate, that is below 2-3 cpm characteristic of a bradygastria condition or slow waves recurring at a higher rate, that is, exceeding 6 cpm to characteristic of a tachygastria condition or other aberrant electrical arrhythmias of the EGG. Typically, such arrhythmias inhibit or delay normal stomach emptying, leading to gastroparesis, nausea, vomiting, and other unpleasant conditions and symptoms identified in U.S. Pat. No. 5,690,691, for example. Thus, implantable monitoring and stimulation systems, sometimes referred to as gastro-intestinal pacemakers, have been proposed in commonly assigned U.S. Pat. Nos. 5,861,014 and 6,216,039, for example to automatically detect such conditions and apply electrical stimulation of the stomach wall to treat such irregular gastric rhythms and restore peristaltic function. Systems have been proposed for artificially pacing the stomach with multiple stimulation pulses applied to in sequential timed sequence to multiple electrode sites, e.g., sites B, C, D of FIG. 1, to induce phased contractions of the stomach and reproduce the normal peristaltic rhythm in order to empty the stomach. A more elaborate system for performing this function involving multiple pairs of electrodes mounted around the stomach wall in a series of electrode arrays and a complex electrical stimulator is disclosed in U.S. Pat. No. 6,243,607. Another elaborate system is disclosed in the above-referenced ′691 patent for artificially pacing the entire GI tract stomach with multiple stimulation pulses that are applied in sequence to multiple sites of the GI tract to reproduce the normal peristaltic rhythm in order to empty the stomach and intestines. The complexity of the circuitry, the battery energy consumption, the invasive surgical procedures to position and attach electrodes at the depicted multiple sites of the ′607 and ′691 patents, the difficulty of attaching multiple electrical medical leads to a housing for the circuitry and battery render these systems impractical at this time.

Returning to treatment of obesity, it has been hypothesized that retaining food in the stomach for a prolonged time promotes a prolonged “full” feeling and discourage further food intake. It was observed that the normal peristaltic rhythm of the EGG could be intentionally disrupted by electrical stimulation applied in the antrum resulting in inhibition or slowing of stomach emptying in animal studies published by S.K. Sarna et al., in “Gastric Pacemakers”, Gastroenterology 70:226-31, 1976. Distal antral stimulation in dogs produced a delay in emptying of liquids and solids. Proximal stimulation was found to have no effect on antral emptying. K. A. Kelly et al. confirmed these findings in their article “Duodenalgastric reflux and slowed gastric emptying by electrical pacing of the canine duodenal pacesetter potential” Gastroenterology, 72:429-33, 1977. Kelly et. al. demonstrated retrograde propulsion of duodenal contents with distal duodenal stimulation and entrainment of the duodenal pacesetter potential.

It has therefore been proposed to treat obesity by interrupting the peristaltic rhythm of the EGG so as to inhibit or slow stomach emptying and prolong a feeling of satiety as described, for example, in U.S. Pat. Nos. 5,423,872 and 5,690,691. The systems disclosed in these patents contemplate implanting gastric pacemakers with one or more stimulation electrodes located so as to stimulate the stomach in a retrograde or reverse phase regime, whereby the induced mechanical contraction of the stomach works against the normal rhythmic stomach contraction caused by the propagation of the slow waves and the higher frequency action potentials depicted in FIG. 1. Thus, it is proposed to stimulate the stomach wall at a rate exceeding the normal peristaltic rate at point C (the ′872 patent FIG. 1) or in reverse phased order at sites D, C and B of FIG. 1 (the ′691 patent FIG. 2).

The electrical stimulation regimens disclosed in the ′872 and ′691 patents involve very wide pulse widths in the range of 10-90 msec in the ′872 patent and 10-1000 msec in the ′691 patent. By contrast, cardiac pacing pulses typically have pulse widths in the range of 0.5 -1.0 msec. Such wide stimulation pulses consume battery energy. Moreover, such wide pulses can create charge imbalances in the tissue-electrode interface that are difficult to dissipate and can lead to elevation of stimulation thresholds, requiring delivery of increased pulse amplitudes and/or electrolytic erosion of the stimulation electrode.

It is also believed that a satiety center in the brain develops the sensation of satiety in a complicated manner believed in part to be due to increased firing of afferent vagal fibers of the vagal nerves extending between the stomach and brain when the stomach is filled. Thus, it has been proposed to electrically stimulate the stomach or the vagus nerves, as set forth in U.S. Pat. Nos. 5,263,480, 5,540,730, and 5,188,104, at a rate mimicking the observed increase to mediate afferent information from the stomach to the satiety centers. Unfortunately, it is not a simple procedure to implant the stomach wall or vagal nerve electrodes, or to do so in an effective place to accomplish the goal of inducing the satiety sensation when the stomach is not actually full. And, the vagal nerves are involved in the regulation of the function of many body organs, including the heart, and stimulation of vagal nerves for any given purpose can have unintended consequences. Moreover, it has been reported that stimulation of the vagal nerves can increase transpyloric flow in pigs in “Vagal Control of Pyloric Resistance”, by C. H. Malbert et al. (Am. J. Physio. 269 (Gastrtointest Liver Physiol 32): G558-569, 1995).

Thus, despite these improvements, there remains a need for treating obesity that is simple to implement and overcomes the disadvantages of the above procedures.

The effects of electrical stimulation of isolated pyloric smooth muscle strips taken from the intermediate sphincter (proximal loop) and the distal pyloric sphincter (distal loop) of human pylorus specimens are reported in the above-cited Schulze-Delrieu et al article. The general conclusion reached was that certain amplitudes and frequencies of applied stimulation induced contraction in the strips taken from the intermediate sphincter for as long as stimulation was applied and relaxation when stimulation was terminated. However, the same stimulation applied to the strips taken from the distal sphincter induced relaxation in about half of the strips.

The effects of directly stimulating the vagal nerves upon pyloric function are described in the above-referenced Malbert et al. article, suggesting that vagal stimulation of at least the left and right and ventral and dorsal vagal nerves at locations superior to the stomach induced relaxation of the pylorus. The effects of “field stimulation” of the duodenum and antrum upon pyloric contraction or activation in animals is reported in the above-referenced Tougas et al. article wherein contraction of the pylorus was reported to have been achieved with field stimulation of the duodenum, although the mechanism was unclear. Such field stimulation of the duodenum may have induced signals in nerves leading to the pylorus. The effects of electrical stimulation of the left greater splanchnic nerve are described in “Pyloric motor response to sympathetic nerve stimulation in dogs” by S. H. Lerman et al. (Surgery, April, 1981 pp. 460-465).

### SUMMARY OF THE INVENTION

The present invention overcomes these disadvantages of the prior art through the selective regulation of the opening and closing of the pylorus lumen to slow or retard stomach emptying following eating to induce a feeling of satiety or to otherwise retain stomach contents or chyme in the stomach for prolonged time periods to thereby limit the patient's desire to eat and to bring about weight loss.

A first aspect of the invention involves slowing or inhibiting the emptying of the stomach contents through delivery of electrical stimulation generated by an implantable gastro-intestinal stimulator into the body that directly or indirectly causes muscle layers of one or both of the intermediate and distal pyloric sphincters to contract and close the pylorus lumen. The implantable gastro-intestinal stimulator preferably comprises a gastro-intestinal stimulation implantable pulse generator (IPG) and pylorus stimulation leads extending from the IPG to a plurality of stimulation electrodes implanted in the muscle layers or about a nerve innervating the muscle layers of the pyloric sphincter causing the muscle layers to contract in response to applied stimulation.

In one particular embodiment of the invention, the pylorus stimulation electrodes are applied directly to or immediately adjacent to the muscles layers of the pyloric sphincters. In another particular embodiment of the invention, the pylorus stimulation electrodes are situated in operative relation to the splanchnic nerve that innervates the pyloric sphincter.

In one operating mode of the invention, stimulation is delivered through the pylorus stimulation electrodes continuously 24 hours per day to decrease the size of the pylorus lumen and retain chyme in the stomach for a longer time to induce a feeling of satiety.

In another operating mode of the invention, stimulation is halted at predetermined times of the day when meals are typically consumed by the patient to enable passage of chyme through the pylorus lumen at that time and stimulation is then resumed to induce a feeling of satiety.

In another operating mode of the invention, the delivery of such electrical stimulation to cause the pylorus to contract and constrict the pyloric lumen is conditioned upon and triggered by the detection of certain GI tract signals, particularly spike potentials characteristic of peristalsis. In this approach, the GI tract signals can be detected by GI tract sensing leads and electrodes and a GI tract signal sense amplifier integrated into the IPG. Or a separate GI tract signal monitor and associated GI tract sensing leads can be implanted in the patient, and telemetry transmissions can be established between the separate IPG and GI tract monitor. A stimulation delay is timed out upon detection of the GI tract signals to enable stomach emptying for a predetermined time, and then stimulation is delivered for a further stimulation duration.

In still another approach, the delivery of such electrical stimulation to cause the pylorus to contract and constrict the pyloric lumen is conditioned upon and triggered by the detection of the ingestion of food through the esophagus during relaxation of the lower esophageal sphincter or the detection of relaxation of the pylorus. Again, a stimulation delay is timed out upon detection of the swallowing or emptying event to enable stomach emptying for a predetermined time, and then stimulation is delivered for a further stimulation duration.

In these latter approaches, the stimulation delay allows the patient to ingest food and the stomach to pass chyme to the duodenum during the stimulation delay, and the pylorus opening is restricted upon time-out of the stimulation delay during the stimulation duration to restrict the pylorus lumen and induce a feeling of satiety.

The parameters of the applied stimulation regimen, the operating modes, and the durations and delays are all made programmable by the attending physician to optimize the efficacy in treating a given patient.

Advantageously, the number of stimulation and sense electrodes is minimized, and the surgical procedure for implanting the electrodes is simple. The stimulation parameters, including pulse amplitude, pulse width and frequency, of stimulation pulses are programmable, and are within ranges that are efficient and avoid adverse polarization effects.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, wherein:

FIG. 1 depicts an example of the peristaltic wave created as GI tract signals, particularly the slow wave and the spike potentials characteristic of peristalsis that can be detected through electrodes coupled to the stomach wall, traverse the stomach wall;

FIG. 2 is a diagrammatic view of a first preferred form of an implantable gastro-intestinal stimulator implanted beneath the skin of a patient applying electrical stimulation directly or indirectly to cause one or both sphincters of the pylorus to contract;

FIG. 3 depicts the pylorus in longitudinal and mucosal section views and showing where stimulation electrodes can be implanted in the muscle layers in relation to the labeled parts of the pylorus;

FIG. 4 is a diagrammatic view of a second preferred form of an implantable gastro-intestinal stimulator implanted beneath the skin of a patient applying electrical stimulation to a splanchnic nerve to indirectly to cause one or both sphincters of the pylorus to contract;

FIG. 5 is a block diagram of the components of the gastro-intestinal stimulation IPG of FIGS. 2 and 4 in relation to an external programmer for programming operating modes and parameters of the IPG for controlling operations of the IPG;

FIG. 6 is a diagrammatic view of a further preferred form of an implantable gastro-intestinal stimulator implanted beneath the skin of a patient with sensing electrodes implanted in the stomach wall and pyloric valve stimulation electrodes implanted in the muscle layers of the pylorus pursuant to FIG. 3;

FIG. 7 is a block diagram of the components of the gastro-intestinal stimulation IPG of FIG. 6 incorporating monitoring circuitry and leads bearing electrodes implanted at selected sites of the stomach wall for developing a GI tract signal characteristic of peristalsis that triggers, in accordance with a further aspect of the invention, delivery of electrical stimulation to the pyloric valve stimulation electrodes implanted in the muscle layers of the pylorus pursuant to FIGS. 2 and 3 or implanted about the splanchnic nerve pursuant to FIG. 4;

FIG. 8 is a diagrammatic view of a further preferred form of an implantable gastro-intestinal stimulator implanted beneath the skin of a patient with impedance sensing electrodes implanted about the esophagus or lower esophageal valve to detect swallowing and trigger stimulation through pyloric valve stimulation electrodes implanted in the muscle layers implanted in the muscle layers of the pylorus pursuant to FIG. 3;

FIG. 9 is a block diagram of the components of the gastro-intestinal stimulation IPG of FIG. 8 incorporating impedance monitoring circuitry for developing impedance signals characteristic of swallowing or opening of the pylorus that triggers, in accordance with a further aspect of the invention, delivery of electrical stimulation to the pyloric valve stimulation electrodes implanted in the muscle layers of the pylorus pursuant to FIGS. 2 and 3 or implanted about the splanchnic nerve pursuant to FIG. 4;

FIG. 10 is a flow chart illustrating the operation of the gastro-intestinal stimulator of FIGS. 2-5 directly or indirectly stimulating the pyloric valve at predetermined times of day or after time-out of a delay time from delivery of a preceding dosage;

FIG. 11 is a flow chart illustrating the operation of the gastro-intestinal stimulator of FIGS. 2-5 directly or indirectly stimulating the pyloric valve at all times except when a command is received from an external programmer or magnet;

FIG. 12 is a flow chart illustrating the operation of the gastro-intestinal stimulator of FIGS. 2-4, 6 and 7 directly or indirectly stimulating the pyloric valve for a period of time triggered by detection of peristalsis; and

FIG. 13 is a flow chart illustrating the operation of the gastro-intestinal stimulator of FIGS. 2-4 and 9 with impedance sense electrodes implanted in the esophageal region to detect swallowing as depicted in FIG. 8 or using the stimulation electrodes implanted in the pylorus region to detect relaxation of the pylorus and directly or indirectly stimulating the pyloric valve upon detection of such swallowing or relaxation.

### DETAILED DESCRIPTION OF THE PREFERRED EMBODIMENTS

In the following detailed description, references are made to illustrative embodiments for carrying out various aspects of the invention.

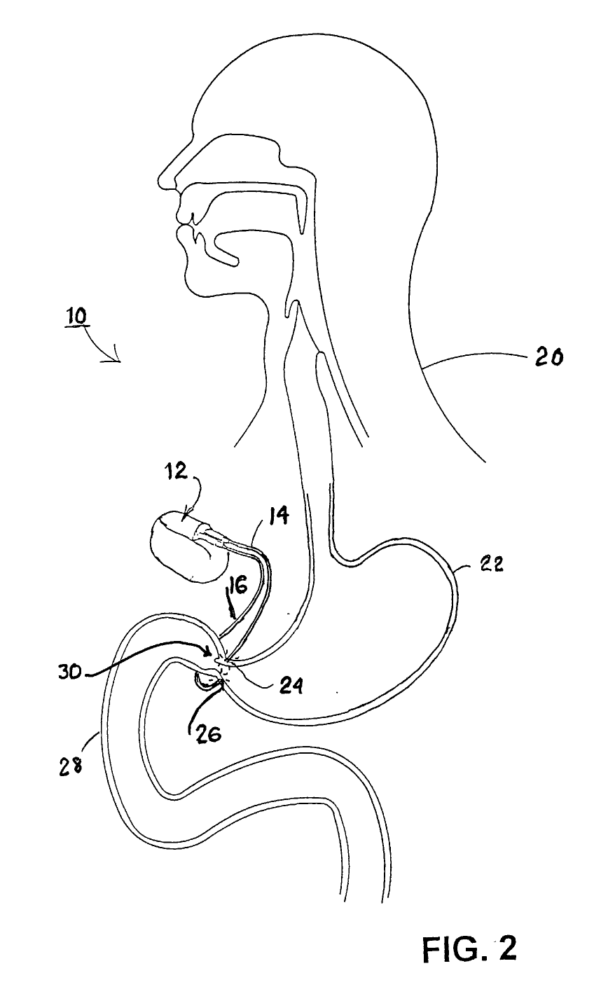

Referring to FIG. 2, an implantable gastro-intestinal stimulator 10 in which the present invention can be practiced is shown implanted in the body of patient 20. The implantable gastro-intestinal stimulator 10 comprises the IPG 12 and electrical stimulation leads 14 and 16 coupled with the IPG 12. The electrical stimulation leads 14 and 16 comprise elongated lead bodies bearing sensing and stimulation electrodes 24 and 26, respectively surgically implanted with respect to the pylorus 30 between stomach 22 and duodenum 28. The electrodes 24 and 26 deliver electrical stimulation generated in IPG 12 across the pylorus 30 that directly or indirectly causes muscle layers of one or both of the intermediate and distal pyloric sphincters to contract and close the pylorus lumen.

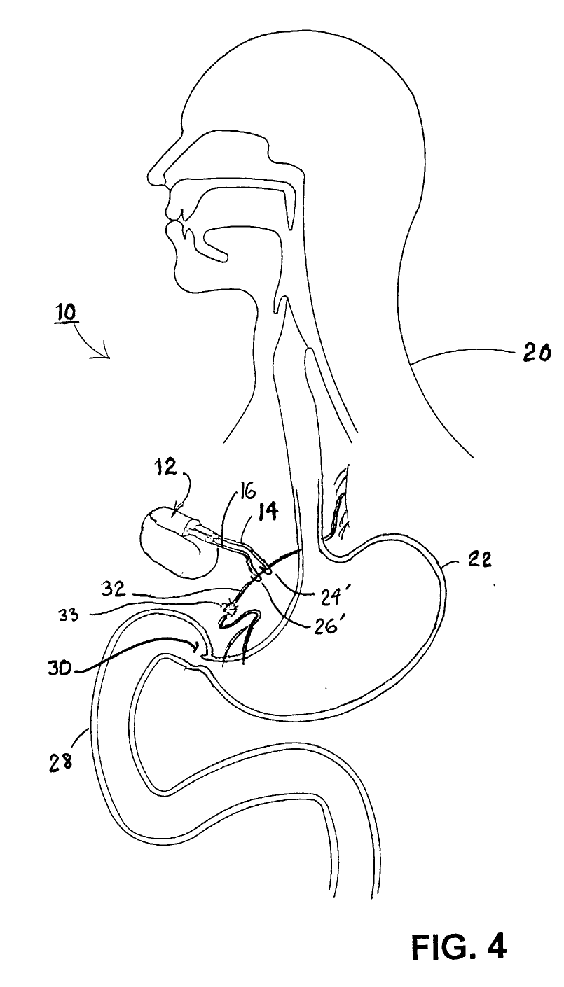

The IPG 12 comprises a hermetically sealed enclosure containing the components depicted in FIGS. 4, 6 or 7 and operating in accordance with a programmed operating mode and programmed operating parameter values as described further below. In one embodiment, the IPG 12 can be of the type represented by the Medtronic® Itrel III Model 7425 IPG, and the leads 14 and 16 pairs of the unipolar Model 4300 or Model 4301 or Model 4351 “single pass” leads available from MEDTRONIC, INC. Such IPGs and leads have been implanted to provide stimulation to sites in the stomach wall to treat chronic nausea and vomiting associated with gastroparesis. The unipolar electrodes of leads 14 and 16 each comprise a length of exposed lead conductor and are of the type disclosed in commonly assigned U.S. Pat. Nos. 5,425,751, 5,716,392 and 5,861,014, for example.

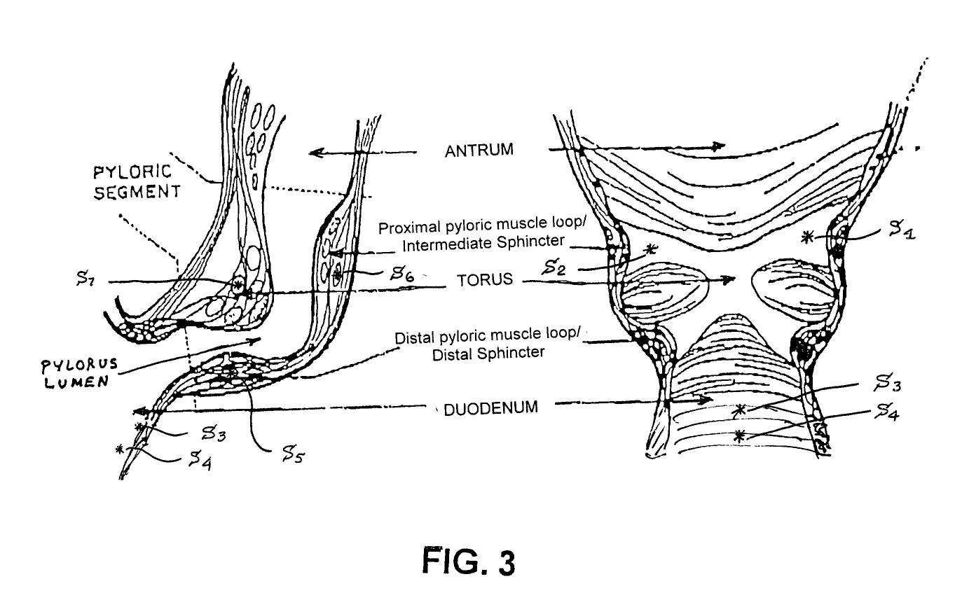

FIG. 3 depicts the pylorus 30 in longitudinal and mucosal section views reproduced from the above-referenced Tougas et al. article and showing where such stimulation electrodes 24 and 26 can be implanted in the muscle layers in relation to the labeled parts of the pylorus 30. Implantation and direct stimulation of the intermediate sphincter at sites S₁ and S₂ may be most efficacious in inducing contraction to narrow or fully close the pylorus lumen.

Alternatively, the electrodes 24 and 26 can be implanted in or against the smooth muscle layers of the duodenum at sites S₃ and S₄ to indirectly stimulate and cause the distal and/or intermediate sphincters to contract to obstruct the pylorus lumen.

The electrodes 24 and 26 can be implanted at other sites for stimulation and sensing, e.g., sites S₆ and S₇. The electrodes 24 and 26 can be implanted at various combinations of the sites S₁ through S₇.

Effective stimulation parameters of a stimulation regimen that induce contraction and the duration of the contraction can be determined during the implantation procedure. Starting parameters can be those described in the above-referenced Schulze-Delrieu et al. article. It will be understood that the stimulation electrodes 24 and 26 at sites S₁ and S₂ can also be used to sense contraction and/or relaxation of the intermediate sphincter through impedance sensing in the manner described in the above-referenced ′730 patent to confirm or monitor the efficacy of contraction following delivery of stimulation intended to effect contraction of the intermediate sphincter.

FIG. 4 is a diagrammatic view of a second preferred form of an implantable gastro-intestinal stimulator 10 implanted beneath the skin of a patient 20 applying electrical stimulation to a splanchnic nerve 32 to indirectly to cause one or both of the intermediate and distal sphincters of the pylorus 30 to contract. The splanchnic nerves are the major nerves supplying sympathetic innervation to the abdomen. The greater, lesser, and lowest (or smallest) splanchnic nerves are formed by preganglionic fibers from the spinal cord which pass through the paravertebral ganglia and then to the coeliac ganglia and plexuses. The lumbar splanchnic nerves carry fibers that pass through the lumbar paravertebral ganglia to the mesenteric and hypogastric ganglia. The greater splanchnic nerve 32 (also called the thoracic splanchnic nerve branches from the thoracic sympathetic ganglion (or trunk) between spinal levels T5 and T9. The splanchnic nerve 32 lies medial to the sympathetic trunk and enters the abdomen by passing through the diaphragm adjacent to the esophagus and terminates in the celiac ganglia and plexus 33. Branch nerve fibers extend from the celiac plexus into the stomach and pancreas.

The IPG 12 is one of the types depicted in FIGS. 4, 6 or 7 that delivers electrical stimulation through the electrodes 24′ and 26′ at the distal ends of leads 14 and 16 respectively and disposed along the splanchnic nerve 32. The nerve stimulation electrodes may take any of the forms known in the art, e.g., the spiral electrodes disclosed in the above-referenced ′480 patent. Effective applied stimulation is expected to be in the range of that stimulation applied in the above-referenced Lerman et al. article and can be determined for each individual patient during the operative procedure. Moreover, in at least one embodiment of the invention, the impedance measuring electrodes can be implanted about or in the sphincters in sites S1 and S2 depicted in FIG. 3 and used to sense contraction and/or relaxation of the intermediate sphincter through impedance sensing in the manner described in the above-referenced ′730 patent to confirm or monitor the efficacy of contraction following delivery of stimulation to the splanchnic nerve intended to effect contraction of one or both of the intermediate sphincter and distal sphincter.

There are a number of ways that the implantable gastro-intestinal stimulator 10 can employed to control the contraction and relaxation of the pylorus in accordance with the various embodiments of the invention. In one approach, stimulation would be delivered to either the muscle layers of the pyloric sphincter in accordance with FIGS. 2 and 3 or the splanchnic nerve in accordance with FIG. 4 continuously 24 hours per day to decrease the size of the pylorus lumen and retain chyme in the stomach for a longer time. In another approach, stimulation would be halted at predetermined times of the day when meals are typically consumed by the patient for a programmed duration to enable passage of chyme through the pylorus lumen at meal times. The interruption of stimulation can be effected either automatically based upon the time of day or a motivated and competent patient can be provided with a means for commanding the interruption for a programmed time duration and a programmed number of times per day associated with eating.

In still further embodiments, stimulation would be delivered to either the muscle layers of the pyloric sphincter in accordance with FIGS. 2 and 3 or the splanchnic nerve in accordance with FIG. 4 to decrease the size of the pylorus lumen and retain chyme in the stomach for a longer time following the automatic detection of a gastro-intestinal response to ingestion of food, e.g., peristalsis or swallowing or stomach emptying. In these embodiments, a stimulation delay would be timed out upon detection of such events to enable passage of some of the stomach contents to the duodenum. Stimulation is delivered for a stimulation duration upon time-out of the stimulation delay to slow further stomach emptying.

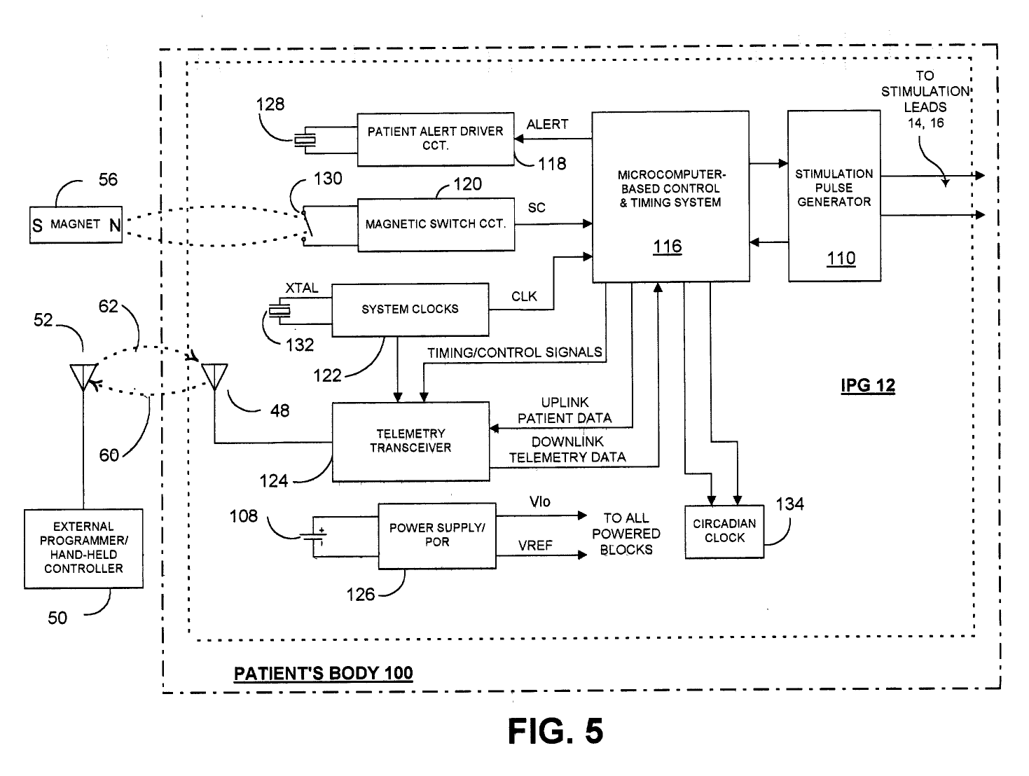

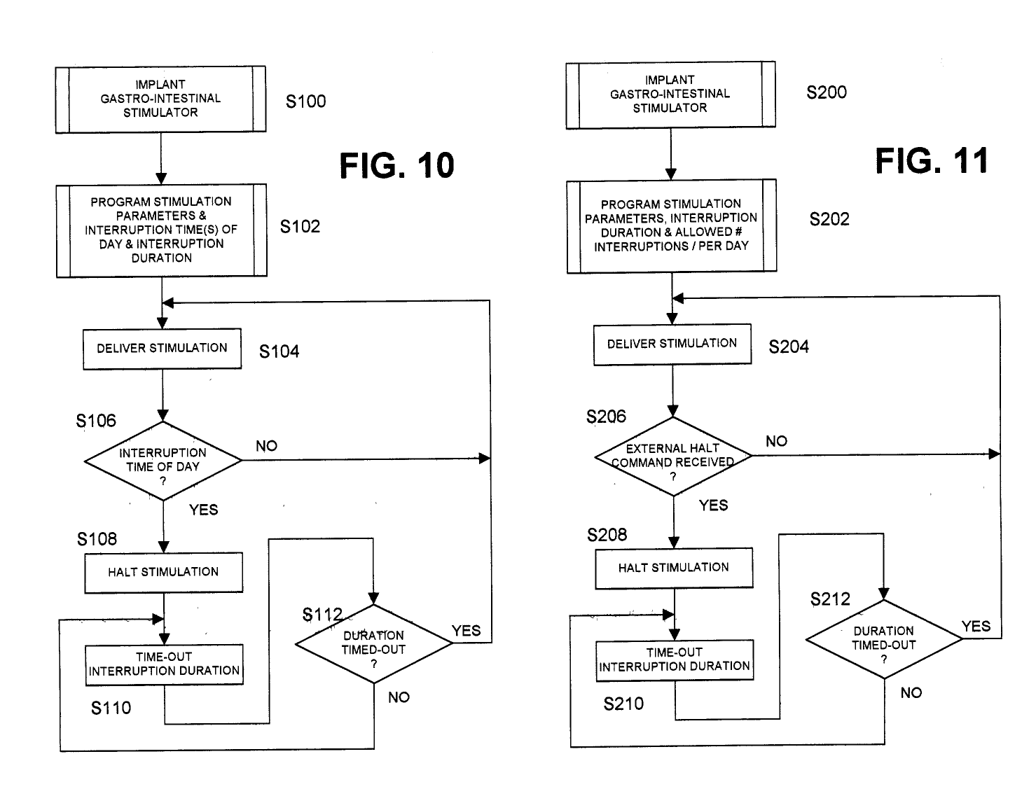

A block diagram of one embodiment of the gastro-intestinal stimulator IPG 12 implanted within a patient's body 100 and in communication with an external programmer 50 and (optionally) an externally applied magnet 56 is depicted in FIG. 5. Flow charts depicting the operation of the gastro-intestinal stimulator IPG 12 of FIG. 5 in these alternative ways are depicted in FIGS. 10 and 11.

The gastro-intestinal stimulator IPG 12 depicted in FIG. 5 has a system architecture that is constructed about a microcomputer-based control and timing system 116 that varies in sophistication and complexity depending upon the type and functional features incorporated therein. The functions of microcomputer-based IPG control and timing system 116 are controlled by firmware and programmed software algorithms stored in RAM and ROM including PROM and EEPROM and are carried out using a CPU, ALU, etc., of a typical microprocessor core architecture.

Power levels and signals are derived by the power supply/POR circuit 126 having power-on-reset (POR) capability from battery(s) 108 to power the electrical circuitry. The power supply/POR circuit 126 provides one or more low voltage power Vlo and one or more VREF sources. Not all of the conventional interconnections of these voltage sources and signals with the circuitry are shown in FIG. 5.

Virtually all current electronic IPG circuitry employs clocked CMOS digital logic ICs that require a clock signal CLK provided by a piezoelectric crystal 132 and system clock 122 coupled thereto. In FIG. 4, each CLK signal generated by system clock 122 is routed to all applicable clocked logic of the microcomputer-based control and timing system 116 and to the telemetry transceiver I/O circuit 124. The system clock 122 provides one or more fixed frequency CLK signal that is independent of the battery voltage over an operating battery voltage range for system timing and control functions and in formatting uplink telemetry signal transmissions in the telemetry I/O circuit 124.

In certain IPGs, an audible patient alert warning or message can be generated by a transducer 128 when driven by a patient alert driver 118 to advise of device operations, e.g., confirmation of delivery of stimulation or interruption of stimulation, or to warn of a depleted battery state.

In the gastro-intestinal stimulator IPG 12, uplink and downlink telemetry capabilities are provided to enable communication with either a remotely located external medical device or programmer 50 or a more proximal medical device on the patient's body or another IMD in the patient's body. For convenience of description, the preferred embodiments are described as follows using RF downlink telemetry (DT) transmissions 62 and uplink telemetry (UT) transmissions 60. The terms “telemeter”, “telemetry transmission” and the like are intended to embrace any action and manner of communicating and conveying patient data and downlink telemetry data between the IPG 12 and any external monitoring device or programmer 50 in the UT direction and the DT direction, respectively.

In an uplink telemetry transmission 60, the external RF telemetry antenna 52 operates as a telemetry receiver antenna, and the IPG RF telemetry antenna 48 operates as a telemetry transmitter antenna. Conversely, in a downlink telemetry transmission 62, the external RF telemetry antenna 52 operates as a telemetry transmitter antenna, and the IPG RF telemetry antenna 48 operates as a telemetry receiver antenna.

The IPG 12 may also include a magnetic field sensor or reed switch 130 and a magnetic switch circuit 120 that develops a switch closed (SC) signal when the switch 128 or other magnetic field sensor responds to an externally applied magnetic field. As a safety feature, current telemetry transmission schemes require the application of a magnetic field to generate the SC signal to enable UT transmission from telemetry transceiver 124 and receipt of DT transmitted commands. But, this requirement is being phased out in favor of high frequency telemetry schemes that can function at greater distances between antennas 52 and 48 and do not employ the magnetic field confirmation of a telemetry session. Such a telemetry scheme is preferably used in the embodiments of the present invention to enable alternative use of the magnet 56 and to enable telemetry communications between the IPG 12 and any other IMDs implanted in the body 100.

The electrical stimulation is generated by the stimulation pulse generator 110 coupled to the stimulation leads 14, 16 under timing and control of the microcomputer-based control and timing system 116 in a manner well known in the art. The electrical stimulation is configured as a pulse or burst of pulses by DT transmitted programming parameters. The pulse can be defined to be a square wave or a ramped or sinusoidal wave having, in each instance, a programmed pulse width and amplitude. Pulses can be delivered continually at a programmed frequency or in bursts of more than one pulse separated by a rest period, whereby a duty cycle is defined. The frequency of the pulses of a burst can also be programmed, and the amplitudes of the last and/or first pulses can be reduced with respect to the remaining pulses of the burst to provide a ramped burst. A stimulation regimen is defined by selection and programming of these pulse parameters.

In addition, a real-time or circadian clock 134 is included in the circuit module 32 driven by system clocks 122 that provides a time of day signal to the microcomputer-based timing and control system 116.

Therefore, in accordance with one embodiment of the present invention depicted in FIG. 10, electrical stimulation is provided through the electrodes 24, 26 or 24′, 26′ during programmed stimulation on-times and may or may not be interrupted automatically depending upon the programmed operating mode. The gastro-intestinal stimulator 10 is implanted in step S100 and programmed in step S102 to deliver programmed stimulation regimens either continually or intermittently. The stimulation regimen includes the pulse amplitude and duration, the frequency of repetition, burst stimulation parameters and any other parameters found to optimally cause the desired contraction of the pylorus lumen.

Thus, either continuous delivery of the stimulation regimen or interruptions at prescribed time(s) of day and for programmed interruption durations can be programmed in step S102. The circadian clock 134 times out the time of day, and the programmed stimulation is delivered by stimulation pulse generator 110 in step S104 until a programmed interruption time of day is detected in step S106. Stimulation is halted in step S108 when a programmed interruption time of day occurs in step S106. The programmed interruption duration is timed out in step S110, and delivery of stimulation is started again in step S104 when the interruption duration times out as determined in step S112. In this way, the physician can program the stimulation delivery to be interrupted for a time, e.g. 30 minutes, at morning breakfast time, lunchtime, and an evening dinnertime.

The stimulation delivered by stimulation pulse generator 110 can be interrupted in other ways as shown, for example, in the flow chart of FIG. 11. A motivated and competent patient can be provided with a magnet 56 that can be applied over the subcutaneously implanted IPG 12 to close switch 130 and prompt of command the control and timing system 116 to interrupt stimulation of the pylorus or splanchnic nerve preceding a meal taken by the patient. Alternatively, the patient could be supplied with a limited function programmer or hand-held controller 50 that the patient could employ to generate a DT transmitted command that is received and interrupt stimulation of the pylorus or splanchnic nerve preceding a meal taken by the patient.

In either case, steps S200-S204 of FIG. 11 are performed in the same manner as steps S100-S104, and stimulation is halted in step S208 when an external interruption or halt command is received as determined in step S206. In step S202, the physician can program the number of times per day that an interruption is accepted or a minimum time between acceptance of a further interruption in step S206 The programmed interruption duration is timed out in step S210, and delivery of stimulation is started again in step S204 when the interruption duration times out as determined in step S212. In this way, the physician can allow the stimulation delivery to be interrupted by the patient for a time, e.g. 30 minutes, at morning breakfast time, lunchtime, and an evening dinner time.

In these embodiments, the eating habits and body weight of the patient would be monitored, and the physician would periodically adjust the stimulation parameters and the interruption durations depending upon the observed response or lack of response.

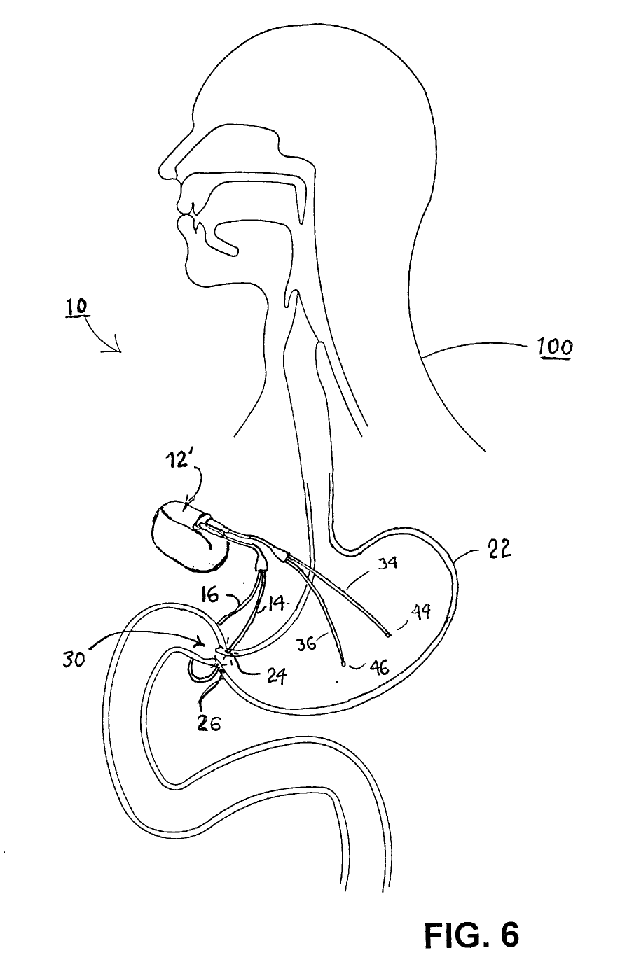

In another approach depicted in FIGS. 6, 7 and 12, the delivery of such electrical stimulation to cause the pylorus to contract and constrict the pyloric lumen is conditioned upon and triggered by the detection of certain GI tract signals, particularly spike potentials characteristic of peristalsis. In this approach, the GI tract signals can be detected by GI tract sensing leads and electrodes and a GI tract signal sense amplifier integrated into the IPG. Or a separate GI tract signal monitor and associated GI tract sensing leads can be implanted in the patient, and telemetry transmissions can be established between a separate IPG 12 and implanted GI tract monitor.

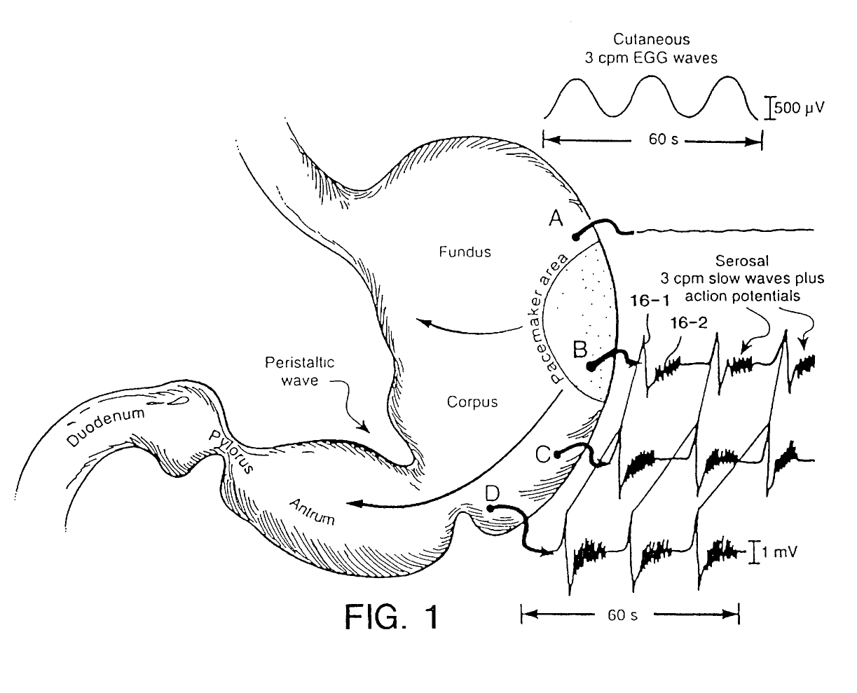

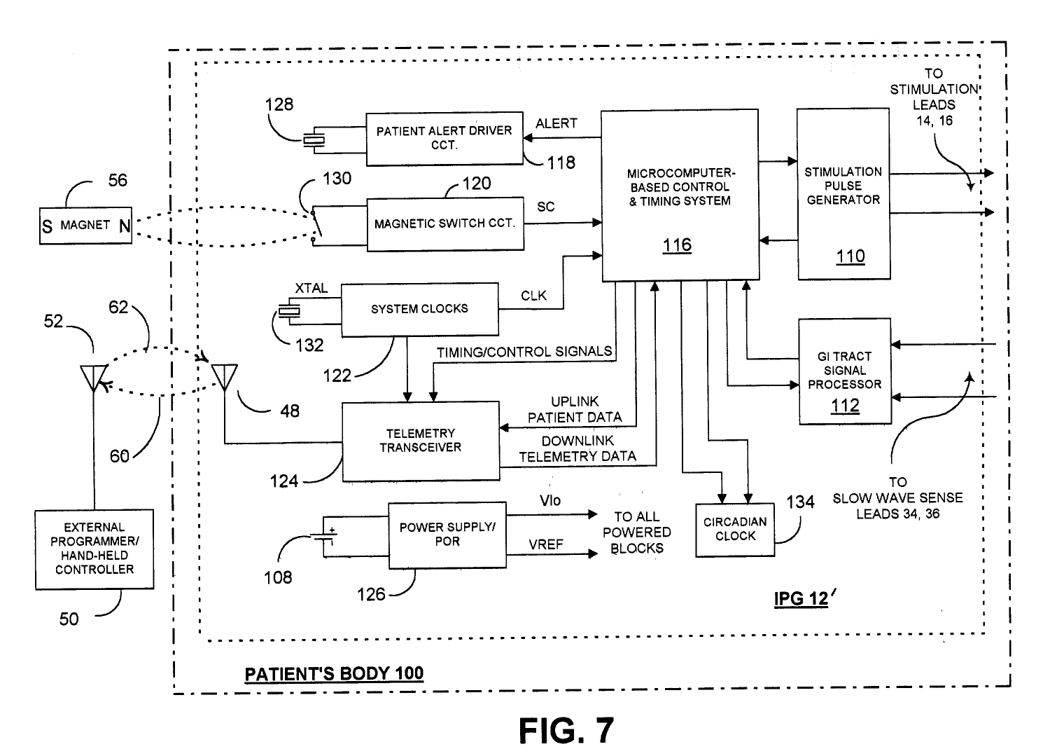

Thus, the gastro-intestinal stimulator IPG 12′ depicted in FIGS. 6 and 7 is modified from the gastro-intestinal stimulator IPG 12 depicted in FIGS. 2-5 to include a GI tract signal processor 112 of the type described in the above-referenced ′249 patent and connector elements for making electrical connection to a pair of GI tract sensing leads 34 and 36. The GI tract sensing leads 34 and 36 have elongated lead bodies enclosing conductors extending to sense electrodes 44 and 46, respectively, at the lead body distal ends that are implanted in the wall of stomach 22 as described in the above-referenced ′249 patent. The GI tract signal processor 112 develops GI tract signals upon detection of spike potentials characteristic of peristalsis described above in reference to FIG. 1. The operating modes of the gastro-intestinal stimulator IPG 12′ depicted in FIGS. 6 and 7 are fully programmable so that IPG 12′ can be programmed to carry about the above-described operating modes or the following operating mode depending upon patient response or failure to respond favorably to any of the operating modes.

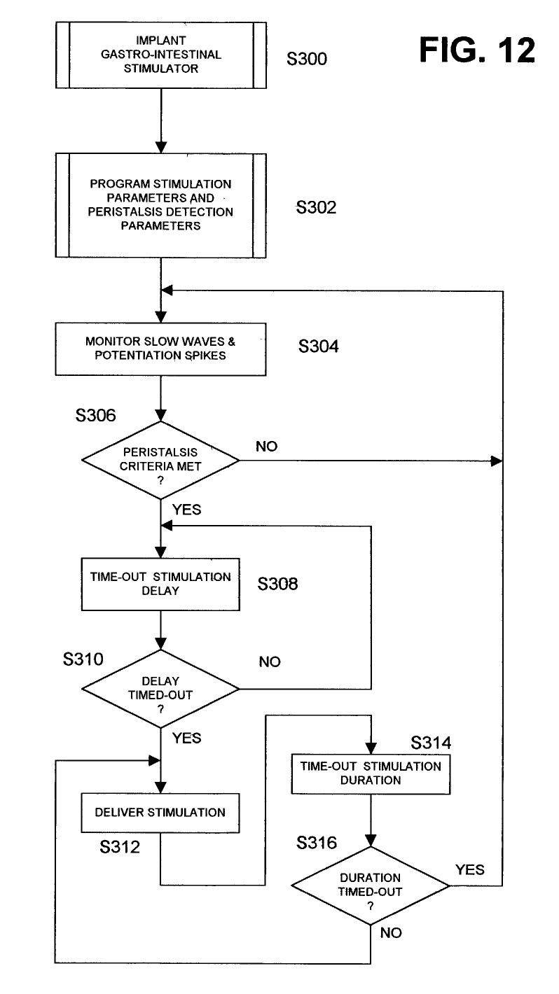

Referring to the operating mode depicted in FIG. 12, steps S300 and S302 are practiced in the same manner as described above with respect to steps S100 and S102 of FIG. 10. The EGG of the stomach is monitored in step S304 by the sense electrodes 44, 46 and the GI tract signal processor 112. The detected GI tract signals are compared to peristalsis criteria in step S306, and peristalsis is declared when the detected GI tract signals satisfy the peristalsis criteria in step S306. It is concluded that the patient is ingesting food when the peristalsis criteria are met. Time-out of a programmable stimulation delay is commenced in step S308 and normal peristaltic wave activity continues during the stimulation delay to both churn the ingested food and allow chyme to pass through the pylorus lumen. Stimulation is delivered to either the muscle layers of the pyloric sphincter in accordance with FIGS. 2 and 3 or the splanchnic nerve in accordance with FIG. 4 to decrease the size of the pylorus lumen in step S312 when the delay times out in step S310. A stimulation duration is timed out in step S314, and stimulation is delivered until the duration times out as determined in step S316.

The programmable stimulation delay timed out in step S308 and the stimulation duration timed out in step S314 are programmable parameters that can be adjusted to optimize the degree to which the patient receives nutrition, demonstrates weight loss, and does not suffer discomfort. In an optimal state, stimulation delay would be set to allow time to pass an adequate amount of nutrition containing chyme and the stimulation during the stimulation duration would induce a feeling of satiety causing the patient to decrease food intake without causing discomfort. It would be expected that the patient would modify and decrease food intake based on experience.

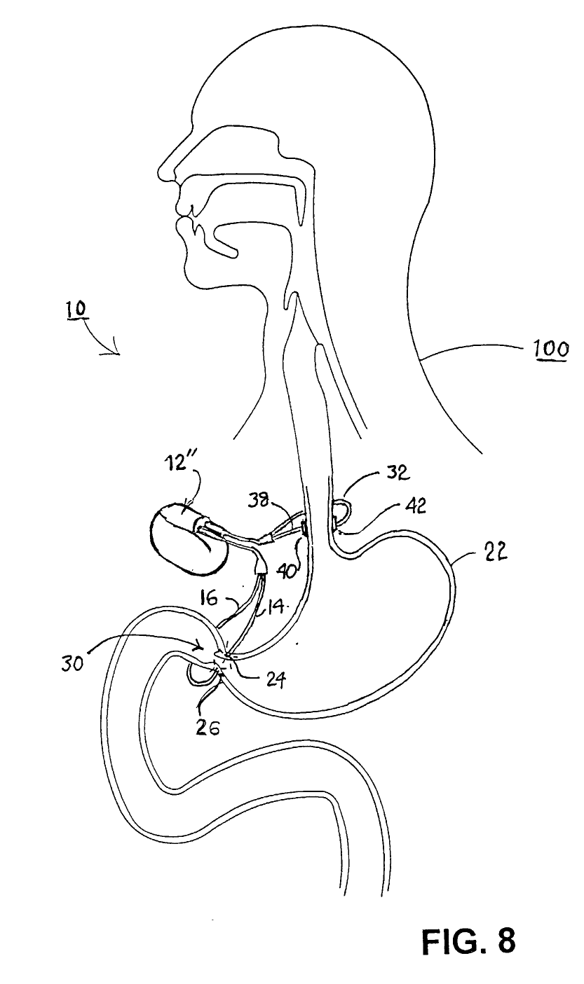

In still another approach illustrated in FIGS. 8, 9 and 13, the delivery of such electrical stimulation to cause the pylorus to contract and constrict the pyloric lumen is conditioned upon and triggered by the detection of the ingestion of food through the esophagus during relaxation of the lower esophageal sphincter or the detection of relaxation of the pylorus. In this approach, impedance signals are developed by an impedance signal processor 114 integrated into the IPG 12 that is coupled to impedance sensing leads and electrodes. Or a separate impedance signal monitor and associated impedance sensing leads can be implanted in the patient, and telemetry transmissions can be established between the separate IPG 12 and such an implanted impedance monitor.

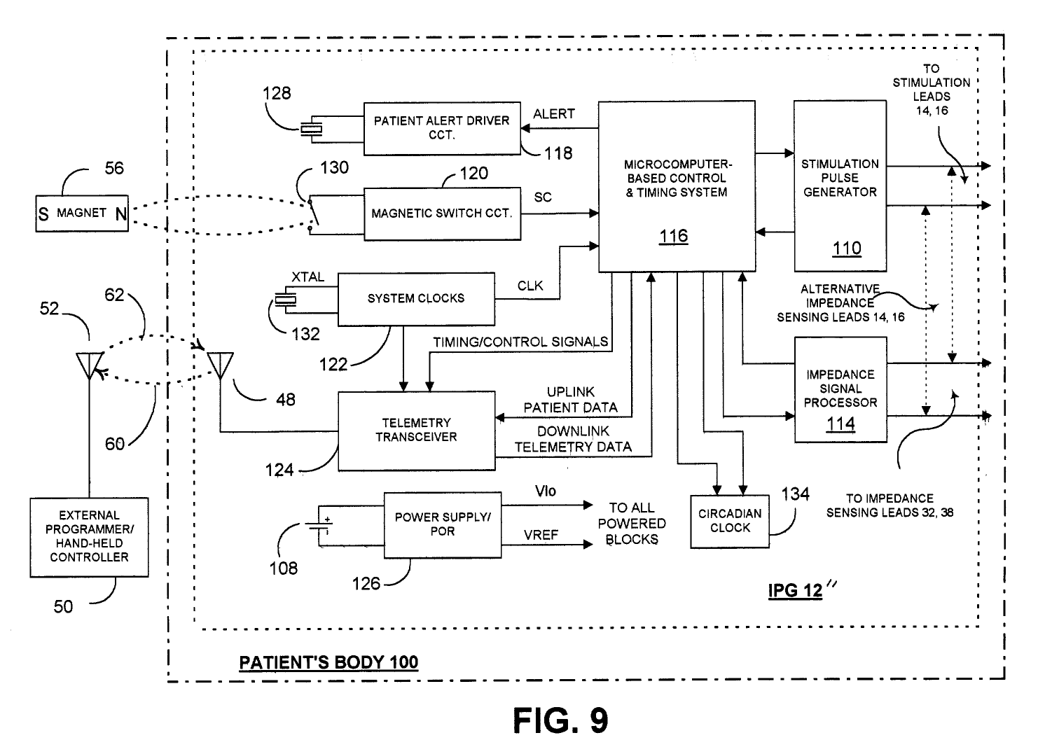

Thus, the gastro-intestinal stimulator IPG 12′ depicted in FIGS. 8 and 9 is modified from the gastro-intestinal stimulator IPG 12 depicted in FIGS. 2-5 to include an impedance signal processor 114 of the type described in the above-referenced ′480 patent and connector elements for making electrical connection to a pair of impedance sensing leads 32 and 38. In the depicted embodiment, the impedance sensing leads 32 and 38 have elongated lead bodies enclosing conductors extending to sense electrodes 42 and 40, respectively, at the lead body distal ends that are implanted to the esophageal wall across the esophagus from one another as described in the above-referenced ′480 patent. The impedance signal processor 114 periodically generates a constant current or voltage between the sense electrodes 42 and 40. A respective measurable voltage or current is developed that is dependent upon the impedance of the tissue between the sense electrodes 42 and 40. The magnitude of the measured voltage or current is measured in the impedance signal processor 114. The measured signal varies as a function of the tissue impedance which itself varies during swallowing. The change in impedance during swallowing of food can be measured during programming of the gastro-intestinal stimulator IPG 12″, and a detection threshold can be developed from the measured impedance change.

In an alternative embodiment, stomach emptying can be determined by coupling the impedance signal processor 114 with the pylorus stimulation electrodes 24 and 26 through the leads 14 and 16, respectively. The contraction and relaxation of the pylorus alters the distance between the stimulation electrodes 24 and 26 resulting in a detectable change in the impedance signal.

The operating modes of the gastro-intestinal stimulator IPG 12″ depicted in FIGS. 8 and 9 are fully programmable so that IPG 12″ can be programmed to carry about the above-described operating modes or the following operating mode depending upon patient response or failure to respond favorably to any of the operating modes.

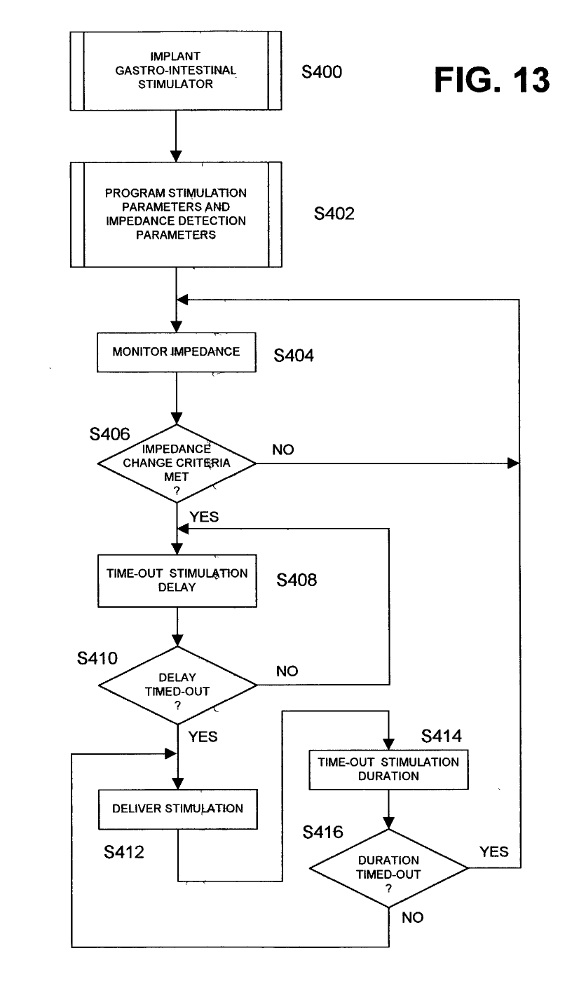

Referring to the operating mode depicted in FIG. 13, steps S400 and S402 are practiced in the same manner as described above with respect to steps S100 and S102 of FIG. 10. The impedance between the electrode pair 40, 42 or the electrode pair 24, 26 is monitored in step S404 by the impedance signal processor 114. The detected impedance signals are compared to swallowing or emptying impedance criteria in step S406. Swallowing or stomach emptying is declared when the detected impedance signal satisfy the swallowing or emptying impedance criteria in step S406. It is concluded that the patient is ingesting food when the swallowing criteria are met or that the patient's stomach is emptying when the emptying criteria are met. As noted above, swallowing and stomach emptying can both be associated with eating.

Time-out of a programmable stimulation delay is commenced in step S408 and normal peristaltic wave activity continues during the stimulation delay to both churn the ingested food and allow chyme to pass through the pylorus lumen. Stimulation is delivered to either the muscle layers of the pyloric sphincter in accordance with FIGS. 2 and 3 or the splanchnic nerve in accordance with FIG. 4 to decrease the size of the pylorus lumen in step S412 when the delay times out in step S410. A stimulation duration is timed out in step S414, and stimulation is delivered until the duration times out as determined in step S416.

Again, the programmable stimulation delay timed out in step S408 and the stimulation duration timed out in step S414 are programmable parameters that can be adjusted to optimize the degree to which the patient receives nutrition, demonstrates weight loss, and does not suffer discomfort. In an optimal state, stimulation delay would be set to allow time to pass an adequate amount of nutrition containing chyme and the stimulation during the stimulation period or duration would induce a feeling of satiety causing the patient to decrease food intake without causing discomfort. It would be expected that the patient would modify and decrease food intake based on experience.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical operation of the above-described devices are not disclosed and are not necessary to the practice of the present invention. In addition, it will be understood that specifically described structures, functions and operations set forth in the above-referenced patents can be practiced in conjunction with the present invention, but they are not essential to its practice.

Thus, embodiments of METHODS AND APPARATUS FOR RETARDING STOMACH EMPTYING FOR TREATMENT OF EATING DISORDERS are disclosed. One skilled in the art will appreciate that the present invention can be practiced with embodiments other than those disclosed. The disclosed embodiments are presented for purposes of illustration and not limitation, and the present invention is limited only by the claims that follow.

1. A method for treating obesity comprising:
supplying electrical stimulation directly or indirectly to the pylorus of a patient in an effective stimulation regimen to substantially close the pylorus lumen to inhibit emptying of the stomach.

2. The method of claim 1, further comprising supplying the electrical stimulation to a nerve innervating a pyloric sphincter of the pylorus.

3. The method of claim 1, further comprising supplying the electrical stimulation directly to a selected site of one of the intermediate and distal sphincters of the pylorus.

4. The method of claim 1, further comprising the steps of:
detecting the commencement of a customary mealtime according to the patient's circadian cycle; and
responding to the detected commencement of the customary mealtime by interrupting supplying of the electrical stimulation for a predetermined interruption duration.

5. The method of claim 1, further comprising the steps of:
detecting a halt command from the patient at the commencement of a mealtime, and
responding to the detected halt command by interrupting supplying of the electrical stimulation for a predetermined interruption duration.

6. The method of claim 1, wherein the supplying step further comprises the steps of:
detecting a peristaltic wave in the stomach of the patient; and
responding to the detected peristaltic wave by supplying the electrical stimulation for a predetermined stimulation duration.

7. The method of claim 1, wherein the supplying step further comprises the steps of:
detecting the relaxation of the pylorus, and
responding to the detected relaxation of the pylorus by supplying the electrical stimulation for a predetermined stimulation duration.

8. The method of claim 1, wherein the supplying step further comprises the steps of:
detecting a swallowing function of the patient accompanying ingestion of food, and
responding to the detected swallowing function by supplying the electrical stimulation for a predetermined stimulation duration.

9. The method of claim 1, wherein the supplying step further comprises the steps of:
detecting a peristaltic wave in the stomach of the patient;
timing a stimulation delay from detection of the peristaltic wave; and
supplying the electrical stimulation for a predetermined stimulation duration upon time-out of the stimulation delay.

10. The method of claim 1, wherein the supplying step further comprises the steps of:
detecting the relaxation of the pylorus,
timing a stimulation delay from detection of the relaxation of the pylorus; and
supplying the electrical stimulation for a predetermined stimulation duration upon time-out of the stimulation delay.

11. The method of claim 1, wherein the supplying step further comprises the steps of:
detecting a swallowing function of the patient accompanying ingestion of food,
timing a stimulation delay from detection of the swallowing function of the patient; and
supplying the electrical stimulation for a predetermined stimulation duration upon time-out of the stimulation delay.

12. The method of claim 1, wherein the supplying step further comprises the steps of:
detecting electrical signals associated with a peristaltic wave in the stomach of the patient; and
responding to the detected peristaltic wave by supplying the electrical stimulation for a predetermined stimulation duration.

13. The method of claim 1, wherein the supplying step further comprises the steps of:
measuring impedance changes of the pylorus associated with relaxation and contraction of the pylorus;
detecting the relaxation of the pylorus from the measured impedance, and
responding to the detected relaxation of the pylorus by supplying the electrical stimulation for a predetermined stimulation duration.

14. The method of claim 1, wherein the supplying step further comprises the steps of:
measuring impedance changes of the esophagus associated with expansion of the esophagus during swallowing;
detecting a swallowing function of the patient accompanying ingestion of food from measured impedance changes of the esophagus, and
responding to the detected swallowing function by supplying the electrical stimulation for a predetermined stimulation duration.

15. The method of claim 1, wherein the supplying step further comprises the steps of:
detecting electrical signals associated with a peristaltic wave in the stomach of the patient;
timing a stimulation delay from detection of the peristaltic wave; and
supplying the electrical stimulation for a predetermined stimulation duration upon time-out of the stimulation delay.

16. The method of claim 1, wherein the supplying step further comprises the steps of:
measuring impedance changes of the pylorus associated with relaxation and contraction of the pylorus;
detecting the relaxation of the pylorus from the measured impedance,
timing a stimulation delay from detection of the relaxation of the pylorus; and
supplying the electrical stimulation for a predetermined stimulation duration upon time-out of the stimulation delay.

17. The method of claim 1, wherein the supplying step further comprises the steps of:
measuring impedance changes of the esophagus associated with expansion of the esophagus during swallowing;
detecting a swallowing function of the patient accompanying ingestion of food from measured impedance changes of the esophagus,
timing a stimulation delay from detection of the swallowing function of the patient; and
supplying the electrical stimulation for a predetermined stimulation duration upon time-out of the stimulation delay.

18. A method of delivering electrical stimulation from an implantable gastro-intestinal tract stimulator comprising an implantable pulse generator and at least one electrical medical lead to suppress appetite, the method comprising the steps of:
surgically implanting at least one electrical stimulation electrode of the electrical medical lead in operative association with the muscle layers of a pyloric sphincter;
coupling the electrical medical lead to the implantable pulse generator; and
operating the implantable pulse generator to deliver electrical stimulation through the implantable medical lead and stimulation electrode to the muscle layers of the pyloric sphincter to cause the muscle layers to contract and close the pylorus lumen to inhibit passage of chyme from the stomach into the duodenum.

19. A method of delivering electrical stimulation from an implantable gastro-intestinal tract stimulator comprising an implantable pulse generator and at least one electrical medical lead to suppress appetite, the method comprising the steps of:
surgically implanting at least one electrical stimulation electrode of the electrical medical lead in operative association with a nerve innervating a pyloric sphincter of a pylorus;
coupling the electrical medical lead to the implantable pulse generator; and
operating the implantable pulse generator to deliver electrical stimulation through the implantable medical lead and stimulation electrode to the nerve innervating the pyloric sphincter to cause the muscle layers of the pyloric sphincter to contract and close the pylorus lumen to inhibit passage of chyme from the stomach into the duodenum.

20. A method of treating patients for obesity comprising the steps of:
delivering electrical stimulation to the patient's body at a site effecting substantial closure of the pylorus lumen;
detecting the commencement of a customary mealtime according to the patient's circadian cycle;
responding to the detected commencement of the customary mealtime by halting delivery of the electrical stimulation to enable relaxation and opening of the pylorus lumen;
timing an interruption duration; and
resuming delivery of the electrical stimulation to the site to effect substantial closure of the pylorus lumen.

21. Apparatus for treating obesity comprising:
an implantable pulse generator adapted to be implanted in a patient's body that develops electrical stimulation in an electrical stimulation regimen; and
at least one lead extending from the implantable pulse generator to supply the electrical stimulation directly or indirectly to the pylorus of a patient in an effective stimulation regimen to substantially close the pylorus lumen to inhibit emptying of the stomach.

22. The apparatus of claim 21, wherein said lead further comprises at least on stimulation electrode adapted to be implanted in operative relation with a nerve innervating a pyloric sphincter of the pylorus for supplying the electrical stimulation to the nerve.

23. The apparatus of claim 21, wherein said lead further comprises at least on stimulation electrode adapted to be implanted in operative relation with one of the intermediate and distal sphincters of the pylorus for supplying the electrical stimulation thereto.

24. The apparatus of claim 21, wherein said implantable pulse generator further comprises:
means for detecting the commencement of a customary mealtime according to the patient's circadian cycle; and
means for responding to the detected commencement of the customary mealtime by interrupting supplying of the electrical stimulation for a predetermined interruption duration.

25. The apparatus of claim 21, wherein said implantable pulse generator further comprises:
means for detecting a halt command from the patient at the commencement of a mealtime, and
means for responding to the detected halt command by interrupting supplying of the electrical stimulation for a predetermined interruption duration.

26. The apparatus of claim 21, wherein said implantable pulse generator further comprises:
means for detecting a peristaltic wave in the stomach of the patient; and
means for responding to the detected peristaltic wave by supplying the electrical stimulation for a predetermined stimulation duration.

27. The apparatus of claim 21, wherein said implantable pulse generator further comprises:
means for detecting the relaxation of the pylorus, and
means for responding to the detected relaxation of the pylorus by supplying the electrical stimulation for a predetermined stimulation duration.

28. The apparatus of claim 21, wherein said implantable pulse generator further comprises:
means for detecting a swallowing function of the patient accompanying ingestion of food, and
means for responding to the detected swallowing function by supplying the electrical stimulation for a predetermined stimulation duration.

29. The apparatus of claim 21, wherein said implantable pulse generator further comprises:
means for detecting a peristaltic wave in the stomach of the patient;
means for timing a stimulation delay from detection of the peristaltic wave; and
means for supplying the electrical stimulation for a predetermined stimulation duration upon time-out of the stimulation delay.

30. The apparatus of claim 21, wherein said implantable pulse generator further comprises:
means for detecting the relaxation of the pylorus,
means for timing a stimulation delay from detection of the relaxation of the pylorus; and
means for supplying the electrical stimulation for a predetermined stimulation duration upon time-out of the stimulation delay.

31. The apparatus of claim 21, wherein said implantable pulse generator further comprises:
means for detecting a swallowing function of the patient accompanying ingestion of food,
means for timing a stimulation delay from detection of the swallowing function of the patient; and
means for supplying the electrical stimulation for a predetermined stimulation duration upon time-out of the stimulation delay.

32. The apparatus of claim 21, wherein said implantable pulse generator further comprises:
means for detecting electrical signals associated with a peristaltic wave in the stomach of the patient; and
means for responding to the detected peristaltic wave by supplying the electrical stimulation for a predetermined stimulation duration.

33. The apparatus of claim 21, wherein said implantable pulse generator further comprises:
means for measuring impedance changes of the pylorus associated with relaxation and contraction of the pylorus;
means for detecting the relaxation of the pylorus from the measured impedance, and
means for responding to the detected relaxation of the pylorus by supplying the electrical stimulation for a predetermined stimulation duration.

34. The apparatus of claim 21, wherein said implantable pulse generator further comprises:
means for measuring impedance changes of the esophagus associated with expansion of the esophagus during swallowing;
means for detecting a swallowing function of the patient accompanying ingestion of food from measured impedance changes of the esophagus, and
means for responding to the detected swallowing function by supplying the electrical stimulation for a predetermined stimulation duration.

35. The apparatus of claim 21, wherein said implantable pulse generator further comprises:
means for detecting electrical signals associated with a peristaltic wave in the stomach of the patient;
means for timing a stimulation delay from detection of the peristaltic wave; and
means for supplying the electrical stimulation for a predetermined stimulation duration upon time-out of the stimulation delay.

36. The apparatus of claim 21, wherein said implantable pulse generator further comprises:
means for measuring impedance changes of the pylorus associated with relaxation and contraction of the pylorus;
means for detecting the relaxation of the pylorus from the measured impedance,
means for timing a stimulation delay from detection of the relaxation of the pylorus; and
means for supplying the electrical stimulation for a predetermined stimulation duration upon time-out of the stimulation delay.

37. The apparatus of claim 21, wherein said implantable pulse generator further comprises:
means for measuring impedance changes of the esophagus associated with expansion of the esophagus during swallowing;
means for detecting a swallowing function of the patient accompanying ingestion of food from measured impedance changes of the esophagus,
means for timing a stimulation delay from detection of the swallowing function of the patient; and
means for supplying the electrical stimulation for a predetermined stimulation duration upon time-out of the stimulation delay.

38. A system for treating patients for obesity comprising:
means for delivering electrical stimulation to the patient's body at a site effecting substantial closure of the pylorus lumen;
means for detecting the commencement of a customary mealtime according to the patient's circadian cycle;
means for responding to the detected commencement of the customary mealtime by halting delivery of the electrical stimulation to enable relaxation and opening of the pylorus lumen;
means for timing an interruption duration; and
means for resuming delivery of the electrical stimulation to the site to effect substantial closure of the pylorus lumen.
